(19) United States
(12) Patent Application Publication
(10) Pub. No.: US 2026/0137396 A1
(43) Pub. Date: May 21, 2026

(54) DOUBLE-SIDED DOME OSTEOTOMY REAMER

(71) Applicant: Rafael Neiman, Loomis, CA (US)

(72) Inventor: Rafael Neiman, Loomis, CA (US)

(21) Appl. No.: 19/046,394

(22) Filed: Feb. 5, 2025

Related U.S. Application Data
(60) Provisional application No. 63/721,914, filed on Nov. 18, 2024.

(51) Int. Cl. A61B 17/16

(52) U.S. Cl. CPC A61B 17/16

(57) ABSTRACT

The improved dual-sided dome osteotomy reamer is intended for the shaping of the bone portions for various orthopedic surgical procedures. The dome osteotomy reamer comprises a generally or substantially hemispherical form and is adapted for attachment to a reamer shaft and/or a reamer adapter in order to be driven in rotation about a cutting axis. The dome osteotomy reamer adequately contours the bone portions to create a polyaxial junction or a convex-concave junction for stability, multiaxial alignment and efficiency.

### CROSS-REFERENCE TO RELATED APPLICATIONS

This application claims the benefit of U.S. Provisional Application No. 63/721,914 entitled “Double-Sided Dome Osteotomy Reamer” filed on Nov. 18, 2024, the disclosure of which is incorporated by reference herein in its entirety.

### TECHNICAL FIELD

The invention relates to methods, devices, and systems for an improved instrument used for modifying bone surfaces during orthopedic surgery. More specifically, the invention relates to methods, devices and systems for an improved reamer instrument to prepare bone surfaces for a variety of orthopedic surgical procedures.

### BACKGROUND OF THE INVENTION

Multi-planar bone deformities are common and need proper surgical correction in patients. Multiplanar bone deformities require extensive surgical planning and precision execution. Surgeons are limited in available tools and techniques to adequately address complex multi-plane deformity corrections.

The current technique for a true dome osteotomy requires multiple sets of instruments that are not designed for this purpose and requires an excessive number of surgical steps to achieve the result. Current technique requires obtaining humeral or femoral head reverse reamers and/or acetabular reamers to be used for a different or non-intended surgical procedure. Acetabular (and some reverse reamers) do not have a self-centering feature, which opens the possibility for off-axis reaming of one or both surfaces. This could lead to a bone gap or translation if an intramedullary device is then introduced for fixation causing malunions or non-unions. If the optimum combination of acetabular and reverse reamers is not chosen, there could be poor fit between the two shaped bone ends. This results in significant soft tissue stripping to perform the current technique. Understanding and planning for a multi-planar correction requires expertise, and slight changes in the plan or execution could lead to inability to properly achieve the correction or desired anatomical alignment and/or lead to future complications such as mal-unions or non-unions.

### BRIEF DESCRIPTION OF THE SEVERAL VIEWS OF THE DRAWINGS

FIGS. 1A-1C depicts various plan views of a first embodiment of a dome reamer assembly;

FIG. 2A depicts an exploded isometric view of the first embodiment dome reamer assembly of FIGS. 1A-1C;

FIG. 2B depicts an exploded isometric view of a second embodiment dome reamer assembly;

FIGS. 3A-3D depicts various plan views of an embodiment of a reamer adapter;

FIGS. 4A-4D depict various plan views of an embodiment of a dome reamer;

FIGS. 4E-4F depict a cross-section and magnified cross-section of the dome reamer of FIGS. 4A-4D;

FIG. 4G depicts a cross-sectional view of different embodiments of the positions and orientations of the cutting elements;

FIGS. 5A-5B depict isometric views of different shape embodiments of the dome reamer of FIGS. 4A-4D;

FIGS. 6A-6E depict different plan views of one embodiment of a method of using the dome reamer or dome reamer assembly; and

FIG. 7A-7B depicts a flowchart of the method of using the dome reamer or dome reamer assembly.

### BRIEF SUMMARY OF THE INVENTION

In one embodiment, the dome osteotomy reamer comprises a hollow body, the hollow body comprises a central axis, a body shape, an outer surface, an inner surface, a plurality of openings, and an adapter opening or orifice; and a first plurality of cutting members or elements, the first plurality of cutting members comprises a first plurality of cutting member shapes, the first plurality of cutting members disposed onto the outer surface of the hollow body adjacent to the plurality of openings in a first direction; and a second plurality of cutting members or elements, the second plurality of cutting members comprises a second plurality of cutting member shapes, the second plurality of cutting members disposed onto the inner surface of the hollow body adjacent to the plurality of openings in a second direction.

In another embodiment, the method of using the dome osteotomy bone reamer comprises the steps of: completing the desired osteotomy to create a first and second portions of a bone; creating a first passageway into a first portion of the bone ; aligning the first and second portions of the bone; creating a second passageway into the second portion of the bone; and contouring a portion of the of the first portion of the bone and the second portion of the bone using the dome osteotomy reamer to create a polyaxial joint.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The dome osteotomy assembly may be used in various surgical applications. Such surgical applications include, but not limited to orthopedic conditions where bone defects are reconstructed, allowing for matching surfaces to be mated, such as allografts or autografts in segmental defects. The dome osteotomy reamer can also be employed at the start of the transport phase of distraction osteogenesis bone transport, as the bone surfaces are matched prior to docking occurs, lessening the bone gaps which can be encountered during the docking phase of transport. Also, the dome osteotomy reamer may used with malunions or non-unions, where the bone portions can be re-shaped and re-aligned into compressible dome surfaces to allow compression without gaps.

There exists a need for the improved double-sided dome osteotomy reamer to be used in osteotomy surgical procedures. The improved double-side dome osteotomy reamer has many advantages, including, but not limited to (1) allowing for correction with a forgiving, compressible set of surfaces without significant bone gaps or defects for early or immediate weight bearing; (2) allowing for correction in different planes; (3) creating a successful, arcuate surfaces with mating “cup;” (4) having an inherent stability of the mated bone portions due to the bone portions mating surfaces; (5) more accurate alignment between bone portions; (6) minimal surgical planning compared to focal dome osteotomy or mathematically directed single-cut osteotomy; (7) reduced number of tools used; and (8) more efficient surgical procedure.

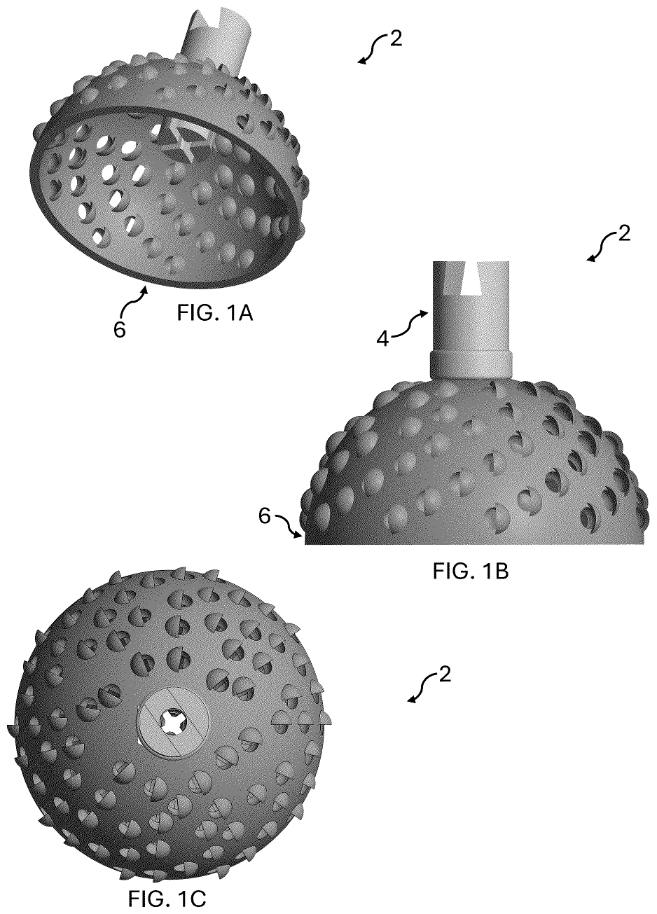

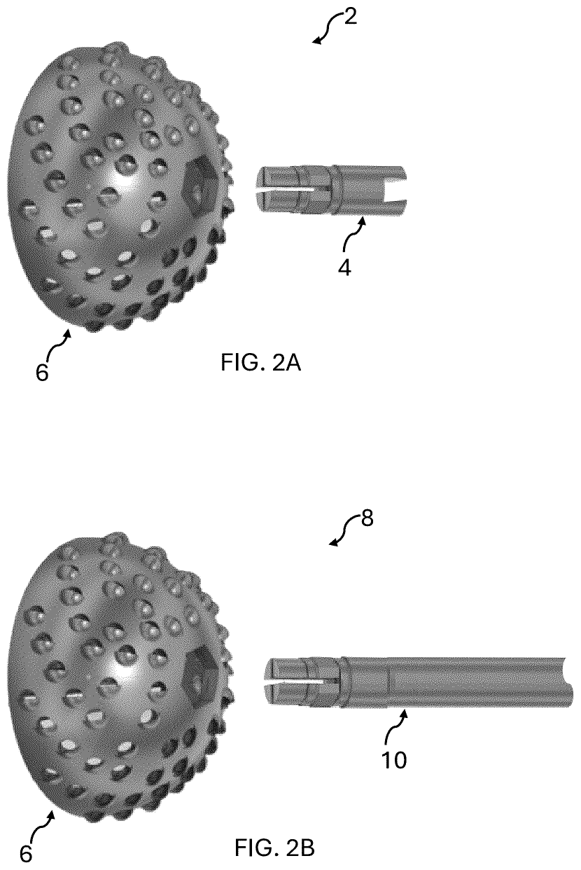

FIGS. 1A-1C and 2A depict various plan views of a first embodiment of a dome osteotomy reamer assembly 2. The dome osteotomy reamer assembly 2 comprises a dome reamer 6 and a reamer adapter 4. The dome osteotomy reamer 6 is removably coupled to the reamer adapter 4. The dome osteotomy reamer 6 is a surgical instrument with dual-sided cutting elements 34, 44 that can cut a concave surface on one end of a first portion of a bone while cutting a matching convex surface on the adjacent portion or second portion of the bone. The activation of the rotational force and a push-and-pull action against one or more portions of the bone to create a polyaxial joint. The polyaxial joint includes a concave surface on a first bone portion and a matching convex surface on a second bone portion that can correct precise combinations of angular and rotational deformities in different planes. The dome osteotomy reamer 6 is significantly less invasive than current instruments. The current process requires a large exposure to deliver each bone end. Standard individual concave acetabular reamers and convex humeral/femoral head reamers require a direct approach to each bone end, whereas the dome osteotomy reamer 6 can be slipped into an osteotomy site with less soft tissue and periosteal stripping, then engaged into a reamer shaft that cuts each bone surface without the need to fully deliver the bone ends.

The reamer adapter 4 is a dual-purpose tool once it is removably coupled to the dome osteotomy reamer 6. The first purpose of the reamer adapter 4 clamps, secures or grips a workpiece through its center with a clamping pressure to keep it immobile and self-centered. The second purpose is to lock the reamer adapter 4 to the dome osteotomy reamer 6 by inserting a workpiece through the center to expand the segmented sleeve arms outwardly to apply a radial force preventing the reamer adapter 4 from loosening. The workpieces may include tools, drill bits, reamers or guide wires. The reamer adapter 4 may be further removably coupled to an existing rotary drill and/or the rotary drill shaft. FIG. 2B depicts a second embodiment of a dome osteotomy reamer assembly 8. The dome osteotomy reamer assembly 8 comprises a dome osteotomy reamer 6 and a reamer rotary drill shaft 10. The rotary drill shaft 10 is also a dual-purpose tool. It includes an elongated shaft that may be directly coupled to an existing rotary drill. The rotary drill may comprise a manual or powered rotary drill.

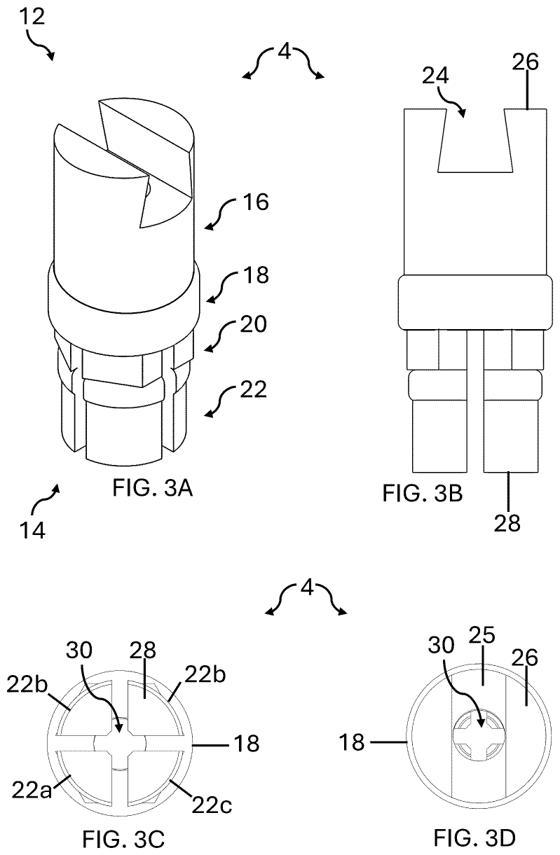

FIGS. 3A-3D depict different plan views of a reamer adapter 4. The reamer adapter 4 comprises a first end 12, a second end 14, a first surface 26 and a second surface 28, a segmented sleeve 22, a connector body 16 and a lumen 30. The lumen 30 extends from the second surface 28 through the channel surface 25. The lumen is sized and configured to receive a surgical workpiece. The surgical workpiece may include drill bits, reamers, guidewires and/or any combination thereof. The segmented sleeve 22 comprises a plurality of segments 22a, 22b, 22c, 22d a coupling band 20. The plurality of segments 22a, 22b, 22c, 22d are arranged spaced apart from each other in a radial pattern. The segmented sleeve 22 may comprise a taper. The coupling band 20 is positioned between the first surface 26 and the second surface. The coupling band 20 comprises a band cross-sectional shape, the band cross-sectional shape may comprise a circle and/or a polygon. In one exemplary embodiment, the band cross-sectional shape comprises a hexagonal cross-sectional shape. The coupling band 20 is sized and configured to engage with the adapter opening 36of the dome osteotomy reamer 6. The coupling band 20 is sized and configured to mate with the adapter opening 36 of the dome osteotomy reamer. The segmented sleeve 22 is positioned near, adjacent and/or within the second end 14. The coupling band 20 may also further comprise segmented band sections.

The connector body 16 comprises collar 18 and a channel 24. The channel 24 extends from a first surface 26 towards the second surface 28. The channel 24 is sized and configured to receive a matching “tail” from an existing rotary drill shaft that is known in the art to form a dovetailed joint. However, other connecting mechanisms may be utilized that are known in the art for coupling and quick release between two or more pieces. The channel 24 comprises a channel surface 25. The connector body comprises a connector body diameter and the collar 18 comprises a collar diameter. The collar diameter of the collar 18 is greater than the connector body diameter. Furthermore, the collar diameter of the collar 18 is greater than a segmented sleeve 22 diameter and/or a coupling band diameter. The increased diameter of the collar 18 allows the reamer adapter 4 to be inserted through the adapter opening 36 of the dome osteotomy reamer 6 and provide a depth stop or a positive stop to prevent the reamer adapter 4 from being inserted through the adapter opening 36 past a desired length or depth.

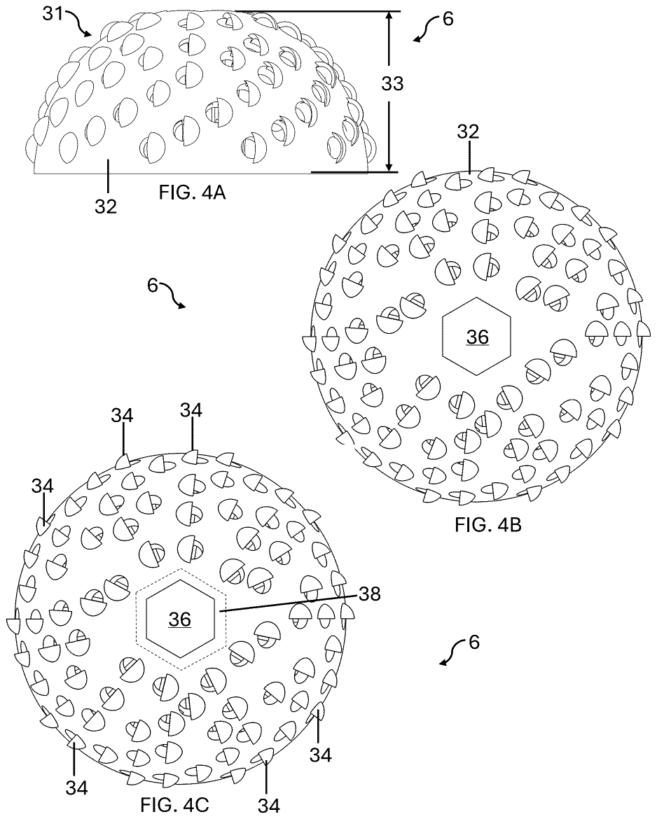

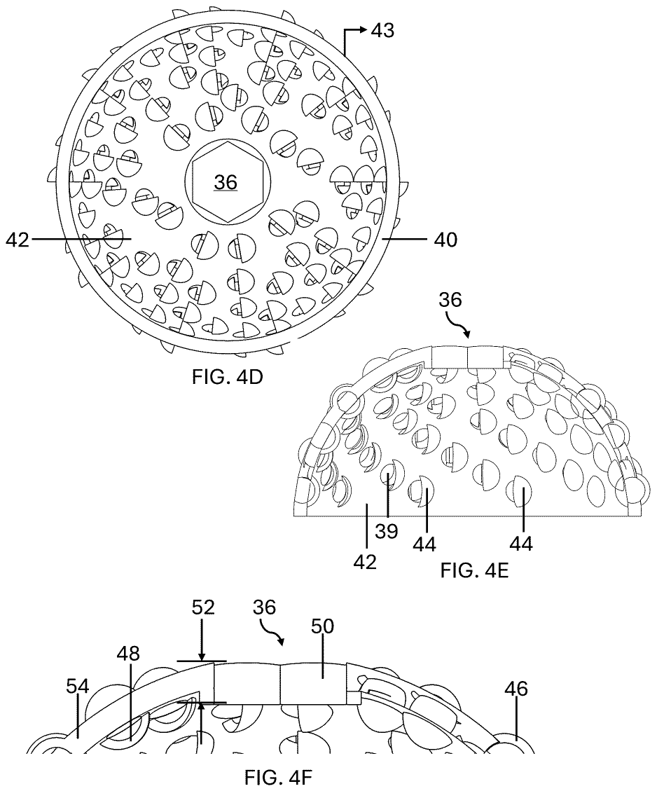

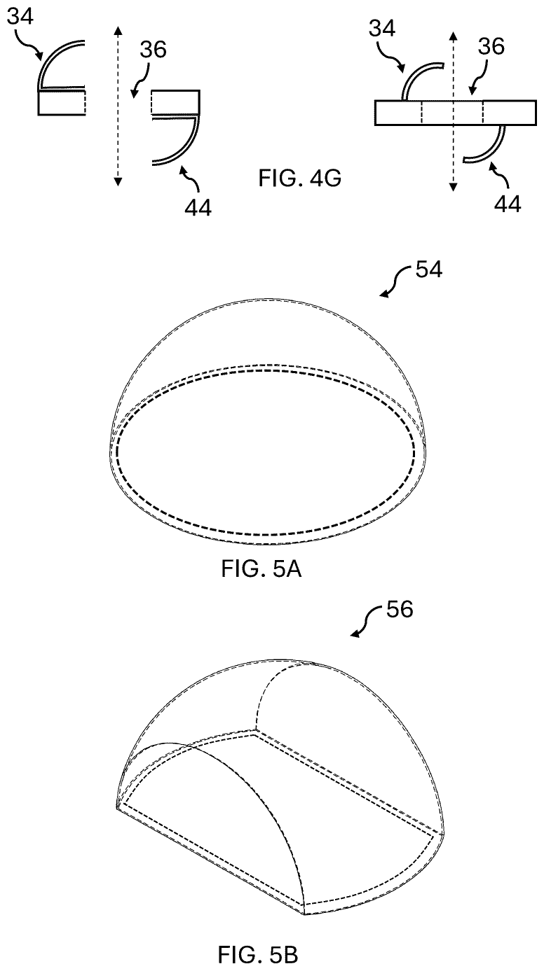

FIGS. 4A-4G depict various plan views of one embodiment of a dome osteotomy reamer 6. The dome osteotomy reamer 6 comprises a hollow body 31 that includes an outer surface 32, an inner surface 42, an opening 36, a plurality of holes 39, a first plurality of cutting elements 34 and a second plurality of cutting elements 44. The hollow body 31 may further include a top surface 38, a hollow body shape and/or a hollow body wall thickness 54.

The plurality of holes 39 are disposed onto the hollow body 31 of the dome osteotomy reamer 6. More specifically, the plurality of holes 39 are disposed circumferentially or radially around the hollow body 31. The plurality of holes 39 are spaced apart from each other. At least a portion of the plurality of holes 31 may comprise a sharpened edge. The plurality of holes 31 extend from the outer surface 32 through the inner surface 42 of the hollow body 31. The plurality of holes 39 allows the small pieces of cut bone and cartilage to pass through the plurality of holes 39 towards the inner surface 42 or outwards towards the outer surface 32 of the hollow body 31 and prevent collection at the surgical site.

The first plurality of cutting elements 34 are disposed onto the outer surface 32 of the hollow body 31 and positioned adjacent and/or over a portion of the plurality of holes 39. The first plurality of cutting elements 34 are positioned in a first cutting direction or first cutting orientation and a first position. The second plurality of cutting elements 44 are disposed onto an inner surface 42 of the hollow body and positioned adjacent to and/or over a portion of the plurality of holes 39. The second plurality of cutting elements 44 are positioned in a second cutting direction or second cutting orientation, and a second position. The first cutting direction is different than the second cutting direction. Alternatively, the first cutting direction is the same as the second cutting direction. The first plurality of cutting elements 34 and the second plurality 44 comprise cutting directions or orientations as mirror images of each other.

The first position of the first plurality of cutting elements 34 is different than the second position of the second plurality of cutting elements 44. Alternatively, the first position is the same as the first position. The second position is 180 degrees apart from the first position. The second position and the first position comprise diametrically opposing positions. The different positions and orientations of the first plurality of cutting elements 34 and the second plurality of cutting elements 44 allow the dome osteotomy reamer 6 to have dual-sided reaming or cutting capabilities without removing the dome osteotomy reamer 6 from the surgical site and risk mating alignment and any complications from malunions or non-unions.

The first plurality of cutting elements 34 and the second plurality of cutting elements 44 comprises a cutting element shape and a cutting surface 46, 48. The cutting element shape comprises a convex, arcuate shape or a dome shape. Alternatively, the cutting element shape comprises a substantially convex, arcuate shape or a dome shape. At least a portion of the cutting surfaces 46, 48 may comprise a sharpened edge.

The adapter opening 36 is sized and configured to receive a portion of the reamer adapter 4 and/or a reamer shaft 10. The adapter opening 36 comprises an adapter opening axis, the adapter opening axis is concentric with a central axis of the dome osteotomy reamer 6. The adapter opening 36 further comprises an opening shape, an inner surface 50, and an opening depth or wall thickness 52. The opening depth or wall thickness 52 is greater than the wall thickness 54 of the hollow body 31. The inner surface 50 will engage, contact and/or mate with the outer surface of the coupling band 20 of the reamer adapter 6 or the reamer shaft 10. The opening shape of the adapter opening 36 may comprise a circle or a polygon shape. In one exemplary embodiment, the opening shape of the opening may comprise a hexagon. The opening shape of the adapter opening 36 may comprise the same shape as the coupling band cross-sectional shape. Accordingly, the opening shape of the adapter opening 36 may match or substantially match the coupling band cross-sectional shape.

The hollow body shape may comprise a convex, hemispherical or dome shape 54, and/or a substantially convex, hemispherical or dome shape 56 as shown in FIG. 5A. The hollow body shape may further comprise a truncated hemispherical or dome shape as shown in FIG. 5B. The truncation may comprise flat or planar sections or surfaces of the hollow body 31. The truncation allows one or more locations to be removed or replaced with the flat or planar sections or surfaces. The truncation may be disposed on a top surface and/or on one or more the sides. In one exemplary embodiment, the truncated portion of the hollow body 31 does not comprise a plurality of holes 39, a first plurality of cutting elements 34 and a second plurality of cutting elements 44 to help minimize damage to soft tissue. In another embodiment, the truncated portion of the hollow body 31 does comprise a plurality of holes 39, a first plurality of cutting elements 34 and a second plurality of cutting elements 44.

The dome osteotomy reamer 6 may be available in different diameters 43 to match or substantially match the width of a patient's bone. The diameters may comprise a range of equal to or greater than 30 mm; a range of equal to or greater than 35 mm; and/or a range of equal to or greater than 40 mm. The diameter may further include a range of 30 mm to 60 mm and/or 35 mm to 55 mm. The diameters may be available in 2 mm, 3 mm or 5 mm increments. The dome osteotomy reamer 6 may comprise a material. The dome osteotomy reamer 6 may be available in different heights 33. The height 33 may be available as small, medium, large, extra-large and/or any other combination. The material may include a polymer, ceramic, a metal, and/or any combination thereof.

### Method of using the Dome Osteotomy Reamer or Reamer Assembly

The following method 76 is a unique and novel method of using the dome osteotomy reamer 6 or the reamer assembly 2,8 for a variety of orthopedic surgical procedures, including, but not limited to multi-planar deformity correction, malunion, non-union, and/or fracture fixation. The method 76 executes the contouring of surfaces of a plurality of bone portions to create a convex-concave junction that is advantageous for multiaxial or multiplanar corrections. Such a method is executed without the use of various additional, unnecessary instruments, as well as the steps are performed more efficiently and reduces or eliminates bone surface mismatch, mating or docking if the cuts are not properly performed. Furthermore, the method 76 can be used in open and/or minimally invasive surgeries. Also, the method 76 may further be used in conjunction with image guided navigational systems, or other systems for precision guiding of the dome osteotomy reamer 6.

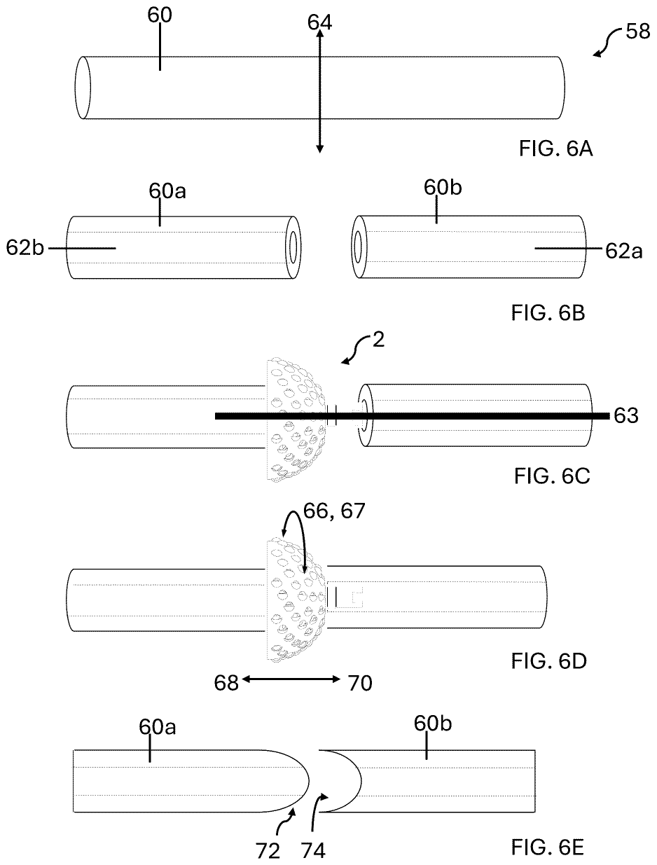

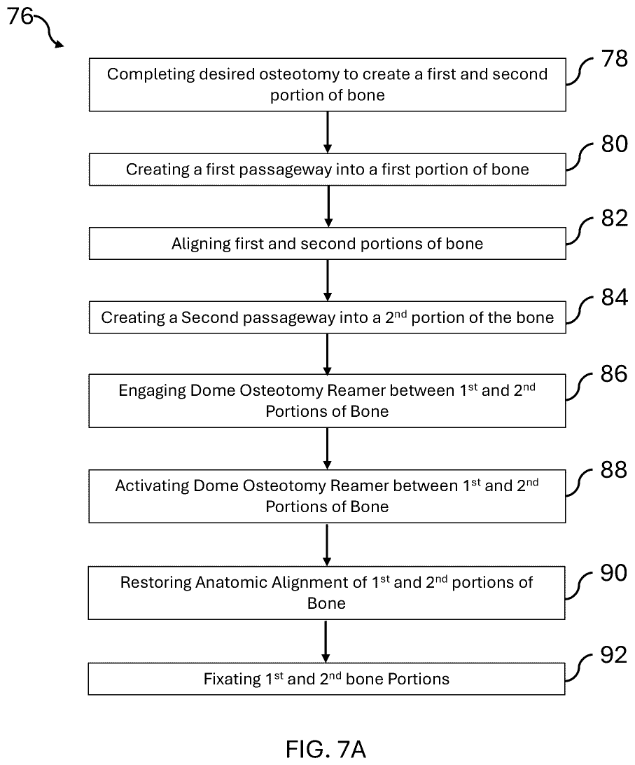

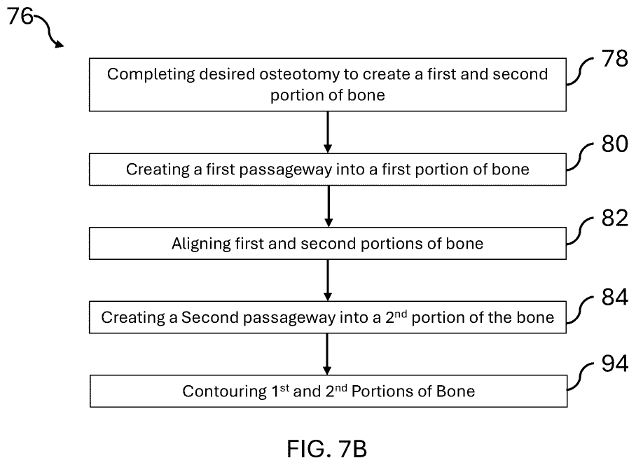

FIGS. 6A-6E and 7A-7B depict at least one embodiment of a method 76 of using the dome osteotomy reamer 6 or reamer assembly 2, 8. In one embodiment, the method 76 of using the dome osteotomy reamer dome osteotomy reamer 6 or reamer assembly 2, 8 comprises the steps of: completing the desired osteotomy to create a first and second portions of a bone 78; creating a first passageway into a first portion of the bone 80; aligning first and second portions of the bone 82; creating a second passageway into a second portion of the bone 84; engaging the dome osteotomy reamer 6 and/or the dome osteotomy reamer assembly 2, 8 between the first and second portions of the bone 86; activating the dome osteotomy reamer 6 and/or the dome osteotomy reamer assembly 88 to create a polyaxial joint.

In another embodiment, the method 76 further includes restoring anatomic alignment 90. In another embodiment, the method 76 further includes restoring anatomic alignment 90 and fixating the first and second bone portions 92. In another embodiment, the method 76 further includes restoring anatomic alignment 90, removing dome osteotomy reamer 6 and/or the dome osteotomy reamer assembly 2,8 (not shown), and fixating the first and second bone portions 92. In another embodiment, the method 76 further includes a preoperative assessment (not shown).

In another alternate embodiment, the method of using the dome osteotomy reamer 6 or reamer assembly 2, 8 comprises the steps of: completing the desired osteotomy to create a first and second portions of a bone 78; creating a first passageway into a first portion of the bone 80; aligning first and second portions of the bone 82; creating a second passageway into a second portion of the bone 84; contouring the first and second portions of the bone by activating the dome osteotomy reamer 6 and/or the dome osteotomy reamer assembly 2, 8 to create a polyaxial joint.

In another embodiment, the alternate method further includes restoring anatomic alignment 90. In another embodiment, the alternate method further includes restoring anatomic alignment 90 and fixating the first and second bone portions 92. In another embodiment, the alternate method further includes removing dome osteotomy reamer 6 and/or the dome osteotomy reamer assembly 2,8 (not shown), restoring anatomic alignment 90, and fixating the first and second bone portions 92. In another embodiment, the alternate method further includes a preoperative assessment (not shown).

The step of completing the desired osteotomy to create a first portion (near side) and second portions (far side) of a bone 78 comprises completing the desired surgical approach and osteotomy 76 of the surgeon. In one embodiment, the osteotomy approach 76 may include a transverse osteotomy. Alternatively, other osteotomy approaches may be used that are known in the art. The desired osteotomy the bone 60 may create a first bone portion 60a and a second bone portion 60b. Accordingly, different osteotomies may be performed for multiple deformity correction to create more than two bone portions.

The step of creating the first passageway 62a into a first portion 60a of the bone 80 may require surgical tools. A rotary drill reamer and/or a rotary drill shaft may be provided to the surgeon or robotic assistant. The rotary drill reamer may be a manual, hand operated tool and/or a power tool. The rotary drill shaft may be coupled to the rotary drill reamer. At least a portion of the rotary drill shaft includes a reamer with cutting elements. A guidewire may be advanced into a first portion of the bone and the rotary drill reamer and drill shaft are inserted over the guidewire to the desired depth is achieved (e.g., equal to greater than 10 mm). The surgeon may activate the rotary drill reamer so that the cutting elements gradually cut and/or remove cartilage and bone in small pieces to create the first passageway. Alternatively, the creation of the passageway may be completed progressively, increasing the reamer size on the drill shaft until a first passageway has been created.

The step of aligning first and second portions of the bone 82 allows precise, centralized passage of the rotary drill reamer and/or the rotary drill reamer shaft. The first portion of the bone (near side) and the second portion of the bone (far side) may be mated or coupled to allow the advancement of a second guidewire 63. The second guidewire is maintained to be coaxial with a central axis of the first and second bone portions. The second guidewire may be advanced across the osteotomy site to ensure that centralized positioning is maintained. Surgeon may confirm with imaging and alignment may be retrograde if required.

The step of creating the second passageway 62b into a second portion 60b of the bone 84 may require surgical tools. A rotary drill reamer and/or a rotary drill shaft may be provided to the surgeon or robotic assistant. The rotary drill reamer may be a manual, hand operated tool and/or a power tool. The rotary drill shaft may be coupled to the rotary drill reamer. At least a portion of the rotary drill shaft includes a reamer with cutting elements. The rotary drill reamer and drill shaft are advanced over the second guidewire to the desired depth is achieved (e.g., equal to greater than 10 mm). The surgeon may activate the rotary drill reamer so that the cutting elements gradually cut and/or remove cartilage and bone in small pieces to create the second passageway. Alternatively, the creation of the passageway may be completed progressively, increasing the reamer size on the drill shaft until a second passageway has been created.

The step of contouring the first and second portions of the bone to create a polyaxial joint 94 may comprise the step of: engaging the dome osteotomy reamer 6 and/or the dome osteotomy reamer assembly 2, 8 between the first 60a and second portions 60 b of the bone 86. The surgeon may engage the dome osteotomy reamer 6 and/or the dome osteotomy reamer assembly 2, 8 with first and second bone portions. The first and second bone portions are distracted and the dome osteotomy reamer 6 and/or the dome osteotomy reamer assembly 2, 8 is positioned between the first and second bone portions. The reamer adapter 4 and/or the reamer shaft 8 are removably coupled together. The second guidewire is engaged with the dome osteotomy reamer 6 and/or the dome osteotomy reamer assembly 2, 8 to ensure that the dome osteotomy reamer 6 and/or the dome osteotomy reamer assembly 2, 8 is centralized or aligned with the central axis of the first and second bone portions and to lock the reamer adapter 4 and/or the reamer shaft 10 to the dome osteotomy reamer 6. The engaging of the second guidewire with the dome osteotomy reamer assembly 2, 8 allows the segmented sleeve portions to expand and prevent withdrawal of the reamer adapter 4 and/or the reamer shaft 10 during high pressure or forces. A first surface of the first bone portion should contact or engage with a portion of the outer surface or a portion of the inner surface of the dome osteotomy reamer 6. Accordingly, the second surface of the second bone portion should contact or engage with a portion of the outer surface or a portion of the inner surface of the dome osteotomy reamer 6.

The step of contouring the first and second portions of the bone to create a polyaxial joint may further comprise the step of: activating the dome osteotomy reamer 6 and/or the dome osteotomy reamer assembly 2, 8 to create a polyaxial joint. The surgeon or robotic assistant may provide a first force 70 against a portion of the first surface of the first portion of the bone. The surgeon or robotic assistant may activate the dome osteotomy reamer 6 and/or the dome osteotomy reamer assembly 2, 8 to begin rotation in a clockwise 66 manner. While in rotation, the first plurality of cutting elements and/or the plurality of holes of the dome osteotomy reamer 6 are cutting bone and cartilage into small pieces which pass through the plurality of holes to be partially contained within the interior of the hollow body 31 of the dome osteotomy reamer 6 or exiting away from the outer surface of the hollow body 31 of the dome osteotomy reamer 6. The first force can create a concave or convex surface in the first bone portion. The first force may include a compression or a tension force (e.g., pushing or pulling).

The surgeon may continue to provide a second force 68 against a portion of the second surface of the second portion of the bone. The surgeon or robotic assistant may activate the dome osteotomy reamer 6 and/or the dome osteotomy reamer assembly 2, 8 to begin rotation in a counterclockwise 67 manner. While in rotation, the second plurality of cutting elements and/or the plurality of holes of the dome osteotomy reamer 6 are cutting bone and cartilage into small pieces which pass through the plurality of holes to be partially contained within the interior of the hollow body 31 of the dome osteotomy reamer 6 or exiting away from the outer surface of the hollow body 31 of the dome osteotomy reamer 6. The second force can create a matching concave or convex surface in the second bone portion. The first force may include compression or tension (e.g., pushing or pulling). The dome osteotomy reamer 6 and/or the dome osteotomy reamer assembly 2,8 (not shown) and/or any other tools may be removed. The first surface on the first portion of the bone can be mated with the second surface on the second portion of the bone to create a polyaxial joint. The concave surface 74 of a first or second bone portion may be mated with the matching convex surface 72 of a first or second bone portion to create a polyaxial joint. Prior to the contouring step, the surgeon may elect to compress the first and second bone portions by using any of the surgical fixation systems known in the art. Any of the dome osteotomy reamers 6 and/or the dome osteotomy reamer assemblies 2, 8 can be used in conjunction with image guided navigational systems, or other systems for precision guiding of the reamers.

The polyaxial joint that is created after contouring, or the convex 72-and-concave 74 junction, is intended to create a larger surface area at the bone junction site to facilitate increased bridging bone and enhanced stability compared with a transverse junction. The convex 72-and-concave 74 nature of the junctional fit also allows for greater degrees of freedom (multiaxial positioning) in reducing the allograft to bone and/or bone-to-bone.

The step of restoring anatomic alignment of the first 60a and the second 60b portions of the bone 90 may be performed using desired techniques known in the art. The step may be used in conjunction with any allografts. Once the soft tissue has allowed for correction and the alignment is achieved using either a universal distractor or other means, compression and fixation can be applied. The step of fixating the first 60a and second 60b bone portions can be performed using desired techniques known in the art. Either internal or external fixation may be considered. Once compression is applied and the fracture is stable, the osteotomy can again be assessed for any bone defects or gaps using imaging. Should there be any critical gaps that may impair fracture healing, the gaps may be grafted with autologous bone graft.

### Example Embodiments

In one embodiment, the bone reamer comprising: a hollow body, the hollow body comprises a central axis, a body shape, an outer surface, an inner surface, a plurality of openings, and an adapter opening or orifice; and a first plurality of cutting members or elements, the first plurality of cutting members comprises a first plurality of cutting member shapes, the first plurality of cutting members disposed onto the outer surface of the hollow body adjacent to the plurality of openings in a first direction; and a second plurality of cutting members or elements, the second plurality of cutting members comprises a second plurality of cutting member shapes, the second plurality of cutting members disposed onto the inner surface of the hollow body adjacent to the plurality of openings in a second direction.

The body shape comprises a generally or substantially hemispherical shape. The body shape comprises a generally or substantially truncated hemispherical shape. The truncation is disposed onto a top surface of the hollow body. The truncation is disposed onto a side surface of the hollow body. The truncation is disposed on at least two opposing sides of hollow body. The top surface of the hollow body comprises a flat, planar surface. At least one side of the hollow body comprises a flat, planar surface.

The first plurality of cutting members is oriented in a first direction and the second plurality of cutting members are oriented in a second direction, the first direction and the second direction are different. The first plurality of cutting members comprises a mirror image of the second plurality of cutting members. The first plurality of cutting members and the second plurality of cutting members are diametrically opposed. The first plurality of cutting members and the second plurality of cutting members are substantially dome shaped.

The bone reamer further comprises a reamer adapter. The bone reamer further comprises a reamer shaft. The bone reamer further comprises an adapter opening. The adapter opening extends through the top surface. The adapter opening of the bone reamer is sized and configured to receive a portion of the reamer adapter. The bone reamer comprises a reamer wall thickness and the adapter opening comprises an opening wall thickness, the opening thickness is greater than the reamer wall thickness. The bone reamer comprises a metal. The hollow body of the bone reamer comprises a diameter of at least 30 mm.

In one embodiment, the bone reamer system comprises a bone reamer. The bone reamer system may further comprise a bone reamer adapter. The bone reamer system may further comprise a bone reamer shaft. The bone reamer system may comprise a bone reamer, bone reamer shaft and a bone reamer adapter. The bone reamer may engage with the bone reamer adapter, the bone reamer adapter engages to the bone reamer shaft. The bone reamer may engage directly to the bone reamer shaft. The bone reamer shaft may be coupled to a rotary drill.

In another embodiment, the method of using the dome osteotomy reamer comprises the steps of completing the desired osteotomy to create a first and second portions of a bone; creating a first passageway into a first portion of the bone; aligning the first and second portions of the bone; creating a second passageway into the second portion of the bone; and

contouring a portion of the of the first portion of the bone and the second portion of the bone using the dome osteotomy reamer to create a polyaxial joint.

The method further includes the steps of: restoring anatomic alignment; and fixating the first and second bone portions of the bone. The step of contouring further comprises the steps of: engaging or positioning the dome osteotomy reamer and/or the dome osteotomy reamer assembly between the first and second portions of the bone; and activating the dome osteotomy reamer. The step of contouring further comprises the step of activating the dome osteotomy reamer. The step of activating the dome osteotomy reamer comprises the steps of: applying a first force against a first surface of the first portion of the bone and rotating the dome osteotomy reamer in a first cutting direction; and applying a second force against a second surface of the second portion of the bone and rotating the dome osteotomy reamer in a second cutting direction.

Alternatively, the step of contouring further comprises the steps of: engaging the dome osteotomy bone reamer between the first and second portions of the bone; and activating the dome osteotomy reamer. The step of contouring and/or the step of activating comprises the steps of: applying a first force against a first surface of the first portion of the bone and rotating the dome osteotomy reamer in a first cutting direction; and applying a second force against a second surface of the second portion of the bone and rotating the dome osteotomy reamer in a second cutting direction. The step of contouring and/or the step of activating may further comprise inserting a reamer shaft through a first and/or second passageway; engaging or positioning the dome osteotomy reamer and/or the dome osteotomy reamer assembly between the first and second portions of the bone; and engaging or coupling the dome osteotomy reamer to the reamer shaft.

The first force and the second force are different. The first force comprises compression and the second force comprises tension. The first cutting direction and the second cutting direction are different. The first cutting direction comprises a clockwise direction and the second cutting direction comprises a counterclockwise direction.

The polyaxial joint comprises a convex surface and a concave surface. The concave surface comprises the first surface of the first portion of the bone or the second surface of the second portion. The convex surface comprises the first surface of the first portion of the bone or the second surface of the second portion of the bone.

I/We claim:

1. The bone reamer comprising:
a hollow body, the hollow body comprises a central axis, a body shape, an outer surface, an inner surface, a plurality of openings, and an adapter opening; and
a first plurality of cutting members or elements, the first plurality of cutting members comprises a first plurality of cutting member shapes, the first plurality of cutting members disposed onto the outer surface of the hollow body adjacent to the plurality of openings in a first direction; and
a second plurality of cutting members or elements, the second plurality of cutting members comprises a second plurality of cutting member shapes, the second plurality of cutting members disposed onto the inner surface of the hollow body adjacent to the plurality of openings in a second direction.

2. The bone reamer of claim 1, wherein the body shape comprises a generally or substantially hemispherical shape.

3. The bone reamer of claim 1, wherein the body shape comprises a generally or substantially truncated hemispherical shape.

4. The bone reamer of claim 3, wherein the truncation is disposed onto a top surface of the hollow body.

5. The bone reamer of claim 1, wherein the first direction and the second direction are different.

6. The bone reamer of claim 1, wherein the first plurality of cutting members and the second plurality of cutting members comprises a mirror image.

7. The bone reamer of claim 1, wherein the first plurality of cutting members and the second plurality of cutting members are diametrically opposed.

8. The bone reamer of claim 1, wherein the first plurality of cutting members and the second plurality of cutting members are substantially dome shaped.

9. The bone reamer of claim 1, wherein the adapter opening of the bone reamer is sized and configured to receive a portion of a reamer adapter.

10. The bone reamer of claim 1, wherein the reamer adapter engages with a portion of a reamer shaft.

11. The method of using the dome osteotomy bone reamer system comprises the steps of:
completing the desired osteotomy to create a first and second portions of a bone;
creating a first passageway into a first portion of the bone;
aligning the first and second portions of the bone;
creating a second passageway into the second portion of the bone; and
contouring a portion of the of the first portion of the bone and the second portion of the bone using the dome osteotomy bone reamer to create a polyaxial joint.

12. The method of claim 11, wherein the method further includes the steps of:
restoring anatomic alignment; and
fixating the first and second bone portions of the bone.

13. The method of claim 11, wherein the step of contouring further comprises the step of:
engaging the dome osteotomy bone reamer between the first and second portions of the bone.

14. The method of claim 11, wherein the step of contouring further comprises the steps of:
engaging the dome osteotomy bone reamer between the first and second portions of the bone; and
activating the dome osteotomy reamer.

15. The method of claim 11, wherein the step of contouring comprises the steps of:
applying a first force against a first surface of the first portion of the bone and rotating the dome osteotomy reamer in a first cutting direction; and
applying a second force against a second surface of the second portion of the bone and rotating the dome osteotomy reamer in a second cutting direction.

16. The method of claim 15, wherein the first force and the second force are different.

17. The method of claim 15, wherein the first force comprises compression and the second force comprises tension.

18. The method of 15, wherein the first cutting direction and the second cutting direction are different.

19. The method of claim 18, wherein the first cutting direction comprises a clockwise direction and the second cutting direction comprises a counterclockwise direction.

20. The method of claim 11, wherein the polyaxial joint comprises a convex surface and a concave surface.
